(19) United States
(12) Patent Application Publication
(10) Pub. No.: US 2009/0030322 A1
(43) Pub. Date: Jan. 29, 2009

(54) ULTRASONIC IMAGING APPARATUS AND ULTRASONIC IMAGING METHOD

(76) Inventors: Chiori Fujiwara, Tokyo (JP); Yayoi Abe, Tokyo (JP); Koji Miyama, Tokyo (JP)

Correspondence Address: Patrick W. Rasche;Armstrong Teasdale LLP, Suite 2600, One Metropolitan Square, St. Louis, MO, 63102, US

(21) Appl. No.: 12/179,442

(22) Filed: Jul. 24, 2008

(30) Foreign Application Priority Data
Jul. 26, 2007 (JP) 2007-193979

(51) Int. Cl. A61B 8/14

(52) U.S. Cl. 600/458

(57) ABSTRACT

An ultrasonic imaging apparatus includes: an image acquisition unit which acquires B-mode image information on an imaging region lying within a subject; a display unit which displays the B-mode image information thereon; a contrast agent inflow start detecting device which detects an inflow timing at which a contrast agent administered to the subject starts to flow in the imaging region, using the B-mode image information; and a post-processing starting device which starts up post-processing conducted after the contrast agent has started to flow in the imaging region, in sync with the inflow timing.

### CROSS REFERENCE TO RELATED APPLICATIONS

This application claims the benefit of Japanese Patent Application No. 2007-193979 filed Jul. 26, 2007, herein incorporated by reference in its entirety.

### BACKGROUND OF THE INVENTION

The subject matter disclosed herein relates to an ultrasonic imaging apparatus which displays a B-mode image of a subject to which a contrast agent is administered.

It has recently been practiced to administer a contrast agent to a subject and image or photograph the administered contrast agent using a B-mode image of an ultrasonic imaging apparatus. The contrast agent comprises liquid containing a large quantity of bubbles. The contrast agent administered to the subject circulates within a body with time. Ultrasound produced from the ultrasonic imaging apparatus at this time is strongly reflected from the contrast agent and the contrast agent is plotted or drawn as a B-mode image high in signal intensity. A time change in the contrast agent that circulates within the body provides an operator with clinically-useful information by determining, for example, a time intensity curve (abbreviated as TIC) (refer to, for example, Japanese Unexamined Patent Publication No. 2006-102030).

After the administration of the contrast agent to the subject, the operator brings an ultrasonic probe into closely contact with the subject and waits for inflow of the contrast agent into a targeted affected part or area. During this period, the operator starts a timer synchronized with the administration of the contrast agent and performs rough settings such as the position and brightness or brightness of an imaging region, etc. while referring to the B-mode image. When the contrast agent flows into the imaging region, the operator tries to further adjust a position to be imaged and brightness and the like while holding the ultrasonic probe and thereby acquire an optimum image.

According to the background art, however, the work of the operator is cumbersome and is not easy to concentrate on the acquisition of the optimum image for the contrast agent. That is, the operator needed to perform, after the administration of the contrast agent, the operations such as the startup of the timer, and the adjustments to the imaging position and brightness and the like with timing provided for inflow of the contrast agent into the imaging region while holding the ultrasonic probe.

In recent years in particular, those from one in which bubbles corresponding to constituent elements arc destroyed by irradiation of ultrasound to one in which they repeat expansion/contraction operations without being destroyed by irradiation of ultrasound have also been used as contrast agents. Each of the contrast agents makes it possible to apply the ultrasound repeatedly and observe a dynamic state in a subject over a long period of time.

On the other hand, it is not preferable to administer the contrast agent to the subject repeatedly upon imaging using the contrast agent. Acquiring the optimum image reliably with once-administration has been required. At this time, the above-described cumbersomeness of work interferes with the acquisition of the optimum image.

In terms of these, how to realize an ultrasonic imaging apparatus capable of, when the contrast agent administered to the subject is observed, making various operations easier and lightening an operator's working load becomes important.

### BRIEF DESCRIPTION OF THE INVENTION

It is desirable that the problem described previously is solved.

An ultrasonic imaging apparatus according to the invention of a first aspect includes an image acquisition unit which acquires B-mode image information on an imaging region lying within a subject, a display unit which displays the B-mode image information thereon, a contrast agent inflow start detecting device which detects an inflow timing at which a contrast agent administered to the subject starts to flow in the imaging region, using the B-mode image information, and a post-processing starting device which starts up post-processing conducted after the contrast agent has started to flow in the imaging region, in sync with the inflow timing.

In the invention according to the first aspect, the contrast agent inflow start detecting device automatically detects inflow timing provided to start the inflow of a contrast agent administered to a subject into an imaging region, using B-mode image information. The post-processing starting device starts up post-processing conducted alter the inflow of the contrast agent in the imaging region is started, in sync with the inflow timing.

An ultrasonic imaging apparatus according to the invention of a second aspect is provided wherein in the ultrasonic imaging apparatus described in the first aspect, the contrast agent inflow start detecting device includes a brightness change detecting device which detects a brightness change that occurs in a B-mode image of the B-mode image information.

An ultrasonic imaging apparatus according to the invention of a third aspect is provided wherein in the ultrasonic imaging apparatus described in the second aspect, the brightness change detecting device sets timing for the brightness change to the inflow timing.

In the invention of the third aspect, the brightness change detecting device detects the inflow of a contrast agent.

An ultrasonic imaging apparatus according to the invention of a fourth aspect is provided wherein in the ultrasonic imaging apparatus described in any one of the first to third aspects, the ultrasonic imaging apparatus includes a region-of-interest setting device which sets a region of interest to the B-mode image in the imaging region, which is displayed on the display unit.

In the invention of the fourth aspect, a region in which the detection of a brightness change is performed is set to the limited optimum position.

An ultrasonic imaging apparatus according to the invention of a fifth aspect is provided wherein in the ultrasonic imaging apparatus described in the fourth aspect, the brightness change detecting device detects a brightness change in the B-mode image in the imaging region or the region of interest.

In the invention of the fifth aspect, a brightness change is detected in either whole region in an imaging region or a specific region of interest.

An ultrasonic imaging apparatus according to the invention of a sixth aspect is provided wherein in the ultrasonic imaging apparatus described in the fifth aspect, the brightness change detecting device includes an average pixel value calculating device which calculates an average pixel value of pixel values contained in the B-mode image.

In the invention of the sixth aspect, an average pixel value is defined as a parameter indicative of a change in brightness.

An ultrasonic imaging apparatus according to the invention of a seventh aspect is provided wherein in the ultrasonic imaging apparatus described in the sixth aspect, the brightness change detecting device determines that the brightness change has occurred, when the average pixel value exceeds a threshold value.

An ultrasonic imaging apparatus according to the invention of an eighth aspect is provided wherein in the ultrasonic imaging apparatus described in the sixth aspect, the average pixel value calculating device includes a recording unit which stores therein a time intensity curve indicative of a time change in the average pixel value.

In the invention of the eighth aspect, reference to the time intensity curve is enabled.

An ultrasonic imaging apparatus according to the invention of a ninth aspect is provided wherein in the ultrasonic imaging apparatus described in any one of the fifth to eighth aspects, the brightness change detecting device includes a maximum pixel value calculating device which determines a maximum pixel value of pixel values contained in the B-mode image information.

In the invention of the ninth aspect, a maximum pixel value is defined as a parameter indicative of a brightness change.

An ultrasonic imaging apparatus according to the invention of a tenth aspect is provided wherein in the ultrasonic imaging apparatus described in the ninth aspect, the brightness change detecting device determines that the brightness change has occurred, when the maximum pixel value exceeds a threshold value.

An ultrasonic imaging apparatus according to the invention of an eleventh aspect is provided wherein in the ultrasonic imaging apparatus described in any one of the fifth to tenth aspects, the brightness change detecting device includes a histogram calculating device which determines a histogram of pixel values contained in the B-mode image information.

In the invention of the eleventh aspect, a histogram is defined as a parameter indicative of a brightness change.

An ultrasonic imaging apparatus according to the invention of a twelfth aspect is provided wherein in the ultrasonic imaging apparatus described in the eleventh aspect, the brightness change detecting device determines that the brightness change has occurred, when a distribution of the histogram changes with time.

An ultrasonic imaging apparatus according to the invention of a thirteenth aspect is provided wherein in the ultrasonic imaging apparatus described in the sixth, ninth and eleventh aspects, the brightness change detecting device includes a switch which selects the average pixel value calculating device, the maximum pixel value calculating device or the histogram calculating device.

In the invention of the thirteenth aspect, a parameter indicative of a change in brightness is selected.

An ultrasonic imaging apparatus according to the invention of a fourteenth aspect is provided wherein in the ultrasonic imaging apparatus described in any one of the first to thirteenth aspects, the ultrasonic imaging apparatus includes a speaker which produces a warning sound.

An ultrasonic imaging apparatus according to the invention of a fifteenth aspect is provided wherein in the ultrasonic imaging apparatus described in the fourteenth aspect, the post-processing starting device includes a warning sound generating device which causes the speaker to produce a warning sound in sync with the inflow timing.

In the invention of the fifteenth aspect, the inflow of a contrast agent is notified to an operator easily.

An ultrasonic imaging apparatus according to the invention of a sixteenth aspect is provided wherein in the ultrasonic imaging apparatus described in any one of the first to fifteenth aspects, the ultrasonic imaging apparatus includes a lamp which emits light for evoking attention.

An ultrasonic imaging apparatus according to the invention of a seventeenth aspect is provided wherein in the ultrasonic imaging apparatus described in the sixteenth aspect, the post-processing starting device includes a lamp lighting device which causes the lamp to emit light in sync with the inflow timing.

In the invention of the seventeenth aspect, the inflow of a contrast agent is notified to the operator easily.

An ultrasonic imaging apparatus according to the invention of an eighteenth aspect is provided wherein in the ultrasonic imaging apparatus described in any one of the first to seventeenth aspects, the post-processing starting device includes a timer which starts counting in sync with the inflow timing.

In the invention of the eighteenth aspect, elapsed time from the inflow of a contrast agent is measured.

An ultrasonic imaging apparatus according to the invention of a nineteenth aspect is provided wherein in the ultrasonic imaging apparatus described in the eighteenth aspect, the post-processing starting device includes a timer display control device which causes the display unit to display time information of the timer in sync with the inflow timing.

In the invention of the nineteenth aspect, elapsed time from the inflow of a contrast agent is displayed.

An ultrasonic imaging apparatus according to the invention of a twentieth aspect is provided wherein in the ultrasonic imaging apparatus described in any one of the first to nineteenth aspects, the post-processing starting device includes a detected information display device which displays information on the average pixel value, information on the maximum pixel value or information on the histogram on the display.

In the invention of the twentieth aspect, information about an average pixel value, a maximum pixel value or a histogram is displayed, and the operator is caused to recognize a change in brightness.

According to the invention, inflow timing provided to allow a contrast agent administered to a subject to flow in an imaging region is automatically detected, and post-processing such as the startup of a timer, which occurs after the inflow of the contrast agent, is started up. It is therefore possible to lighten an operator's working load, by extension, allow an operator to concentrate on acquisition of a B-mode image containing the contrast agent and acquire a suitable B-mode image with one contrast-agent administration.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing an overall construction of an ultrasonic imaging apparatus.

FIG. 2 is a block diagram illustrating a functional construction

of a controller.

FIG. 3 is a flowchart showing the operation of the ultrasonic imaging apparatus according to an embodiment 1.

FIG. 4 is an explanatory diagram illustrating one example of a B-mode image to which a region of interest is set.

FIG. 5 is a flowchart showing the operation of a contrast agent inflow start detecting process according to the embodiment 1.

FIG. 6 is an explanatory diagram showing one example of a TIC (Time Intensity Curve).

FIG. 7 is a flowchart showing the operation of a post-processing starting process according to the embodiment 1.

FIG. 8 is an explanatory diagram illustrating a construction of a display unit according to the embodiment 1.

FIG. 9 is a flowchart showing the operation of a contrast agent inflow start detecting process according to an embodiment 2.

FIGS. 10(A), 10(B), and 10(C) are explanatory diagrams showing one example illustrative of histograms and a difference histogram according to the embodiment 2.

### DETAILED DESCRIPTION OF THE INVENTION

Best modes for carrying out ultrasonic imaging apparatuses according to the invention will be explained below with reference to the accompanying drawings. Incidentally, the invention is not limited thereby.

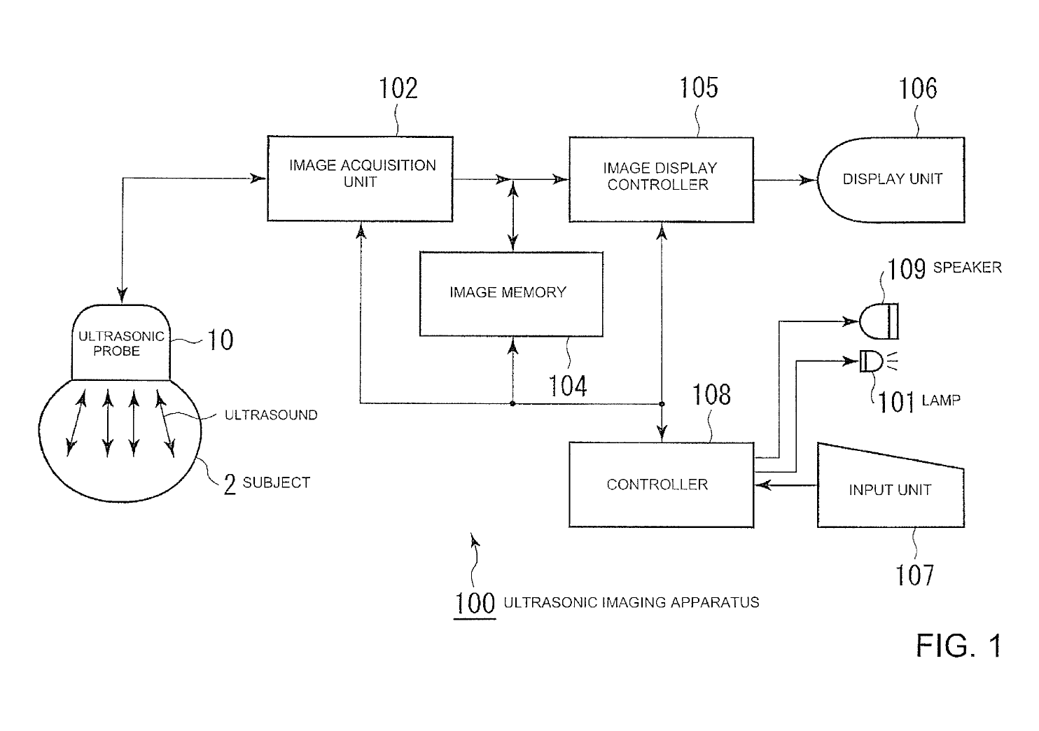

An overall construction of an ultrasonic imaging apparatus 100 according to an embodiment 1 will first be explained. FIG. 1 is a block diagram showing the overall construction of the ultrasonic imaging apparatus 100 according to the embodiment 1. The ultrasonic imaging apparatus 100 includes an ultrasonic probe 10, an image acquisition unit 102, an image memory 104, an image display controller 105, a display unit 106, an input unit 107, a speaker 109, a lamp 101 and a controller 108.

The ultrasonic probe 10 applies ultrasound in a specific direction of an imaged section of a region, i.e., a subject 2 for transmitting and receiving the ultrasound and receives ultrasonic echoes reflected in each case from the inside of the subject 2 as time-series sound rays. The ultrasonic probe 10 includes a probe array in which piezoelectric devices are arranged in array form, and acquires two-dimensional tomographic image information including an electronic scanning direction faced in the direction of this arrangement.

The image acquisition unit 102 includes a transmit-receive part and a B-mode processor or the like. The transmit-receive part is connected to the ultrasonic probe 10 by a coaxial cable and generates an electric signal for driving each piezoelectric device of the ultrasonic probe 10. The transmit-receive part also performs first-stage amplification on each reflected ultrasonic echo received thereat.

The B-mode processor performs a process for generating, in real time, a B-mode image from a reflected ultrasonic echo signal amplified by the transmit-receive part.

The image memory 104 is a large-capacity memory that stores B-mode image information or the like acquired at the image acquisition unit 102. The image memory 104 is constituted of, for example, a hard disk or the like.

The image display controller 105 performs display frame rate conversion of the B-mode image information or the like generated at the B-mode processor and control on the shape and position of an image display.

The display unit 106 includes a CRT (Cathode Ray Tube) or an LCD (Liquid Crystal Display) or the like and displays the B-mode image or the like thereon.

The input unit 107 includes a keyboard, a mouse and the like and is inputted with an operation or control input signal by an operator. The input unit 107 performs, for example, an operation input for selecting the display of a doppler process, designation made by a cursor or the like for performing image processing on displayed image information, an operation input for performing the settings of various threshold values, etc.

The controller 108 includes an image acquisition control unit which controls the operations of the respective parts of the ultrasonic imaging apparatus, based on the operation input signal inputted from the input unit 107 and programs and data stored in advance, and an image processor which performs image processing using the two-dimensional tomographic image information stored in the image memory 104.

The lamp 101 emits light according to instructions given from the controller 108. Upon this light emission, such a color that the operator takes interest therein, e.g., a red color or the like is used.

The speaker 109 produces sound according to instructions issued from the controller 108. This sound produces such a warning sound that the operator takes interest therein,

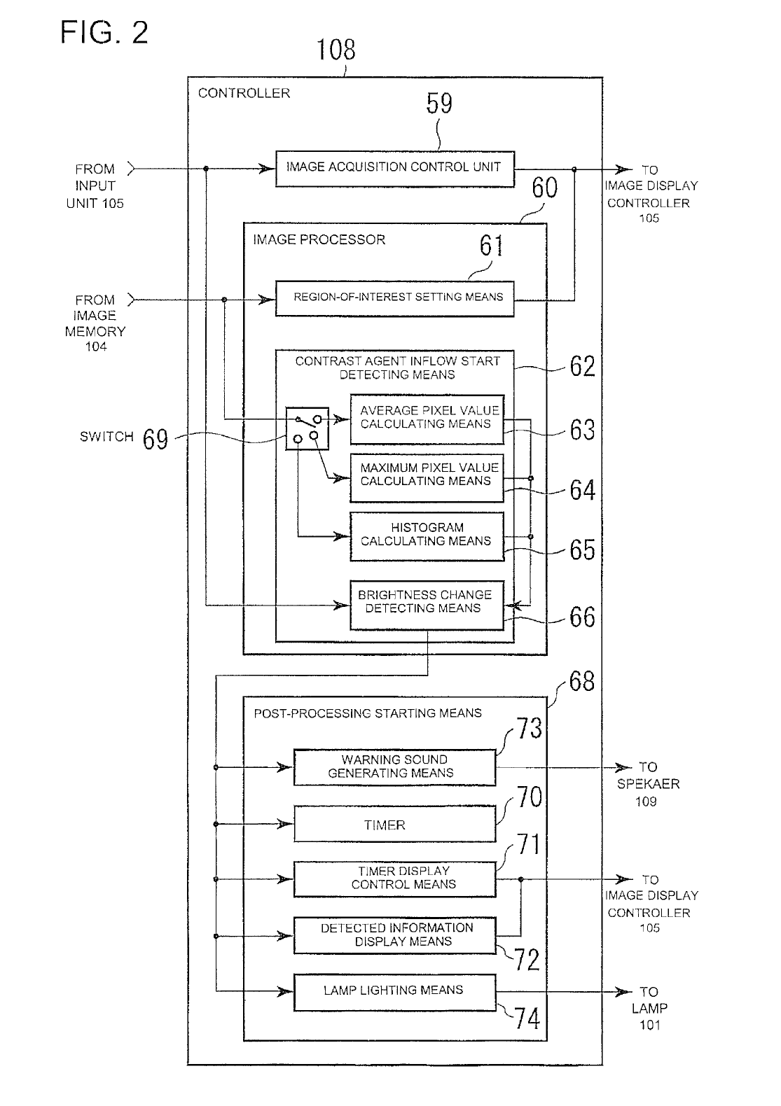

FIG. 2 is a block diagram showing a functional construction of the controller 108. The controller 108 includes an image acquisition control unit 59, an image processor 60 and a post-processing staring means 68.

The image acquisition control unit 59 controls the acquisition of B-mode image information about the subject 2 and performs the display of the acquired B-mode image information and control on the storage thereof in the image memory 104 and the like.

The image processor 60 performs operational or computational processing or the like using the B-mode image information of the image memory 104 and automatically detects the timing provided to allow a contrast agent administered into the subject 2 to flow into a targeted imaging region. The image processor 60 includes a region-of-interest setting means 61 and a contrast agent inflow start detecting means 62.

The region-of-interest setting means 61 sets a region of interest (ROI) to the corresponding B-mode image displayed on the screen of the display unit 106. The region of interest is obtained by designating the position of a cursor existing on the screen using the mouse or the like of the input unit 107. For example, a circular region of interest is set as the region of interest. The region-of-interest setting means 61 extracts the two-dimensional tomographic image information of the image memory 104 corresponding to the set region of interest and transmits the same to the contrast agent inflow start detecting means 62.

The contrast agent inflow start detecting means 62 includes an average pixel value calculating means 63, a maximum pixel value calculating means 64, a histogram calculating means 65, a brightness change detecting means 66 and a switch 69. The average pixel value calculating means 63 takes an average of pixel values of two-dimensional tomographic image information on the set region of interest or two-dimensional tomographic image information containing all imaging regions and determines an average pixel value. Assuming now that a pixel value is Ai, a parameter of a pixel position is i, and the number of pixels in a region of interest or an imaging region is N, an average pixel value AV is calculated by the following equation:

AV=(ΣAi)/N

Incidentally, parameters of Σ indicative of addition are expressed in i=1 to N and omitted in the equation. Incidentally, the average pixel value calculating means 63 has an unillustrated recording unit. Information about the determined average pixel value is stored therein with time series. The information of the recording unit forms information on a TIC (Time Intensity Curve).

The maximum pixel value calculating means 64 determines the maximum pixel value of the pixel values of the two-dimensional tomographic image information on the set region of interest or the two-dimensional tomographic image information containing all imaging regions. Assuming now that a pixel value is Ai, a parameter of a pixel position is i and the number of pixels in a region of interest or an imaging region is N, the maximum pixel value AM is calculated by the following equation:

AM=Max(A1, A2, . . . , An)

Incidentally, the histogram calculating means 65 will be described in detail later.

The switch 69 selects any one of the average pixel value calculating means 63, the maximum pixel value calculating means 64 and the histogram calculating means 65 in response to an input signal sent from the input unit 107. The operator determines whether the two-dimensional tomographic image information is one only for the region of interest or contains all the imaging regions and further selects an optimum detecting method depending on, for example, whether an object to be examined by a contrast agent is in blood or tissue.

The brightness change detecting means 66 detects a change in the brightness of a B-mode image using information on the average pixel value, maximum pixel value or histogram or the like calculated by the average pixel value calculating means 63, maximum pixel value calculating means 64 or histogram calculating means 65 and generates a brightness change detection signal.

The brightness change detecting means 66 is inputted with threshold information from the input unit 107 in advance. When the inflow of a contrast agent into the corresponding imaging region or region of interest is started, the average pixel value or the maximum pixel value increases. The threshold information is used as a reference value for making a decision as to whether the inflow of the contrast agent has been made. When the average pixel value or the maximum pixel value exceeds the threshold value of the threshold information, it is determined that the contrast agent has flowed in. The timing provided to make this decision is defined as inflow timing. Incidentally, the threshold information is decided experimentally in consideration of a detecting method, a region to be imaged and the like.

The post-processing starting means 68 starts post-processing executed after the contrast agent has flowed in the imaging region, with the brightness change detection signal generated by the brightness change detecting means 66 as a startup signal. In the post-processing staring means 68 shown in FIG. 2, an example is shown in which the generation of a warning sound, the lighting of the lamp, the startup of a timer and the display of an indicator are carried out. The post-processing starting means 68 includes a warning sound generating means 73, a lamp lighting means 74, a timer 70, a timer display control means 71 and a detected information display means 72.

The warning sound generating means 73 causes the speaker 109 to produce a warning sound in sync with the brightness change detection signal outputted from the brightness change detecting means 66.

The lamp lighting means 74 causes the lamp 101 to emit light in sync with the brightness change detection signal outputted from the brightness change detecting means 66.

The timer 70 starts the measurement of time from zero in sync with the brightness change detection signal outputted from the brightness change detecting means 66. The timer display control means 71 starts the display of time information being measured by the timer 70 in sync with a detection pulse sent from the brightness change detecting means 66. The timer display control means 71 transmits the time information of the tinier 70 to the image display controller 105 in real time and displays the same on the display unit 106.

The detected information display means 72 displays detected information about the average pixel value, maximum pixel value, time intensity curve, histogram and the like in sync with the brightness change detection signal outputted from the brightness change detecting means 66. Incidentally, in the present embodiment 1, the detected information display means 72 displays an indicator indicative of the average pixel value on the display unit 106.

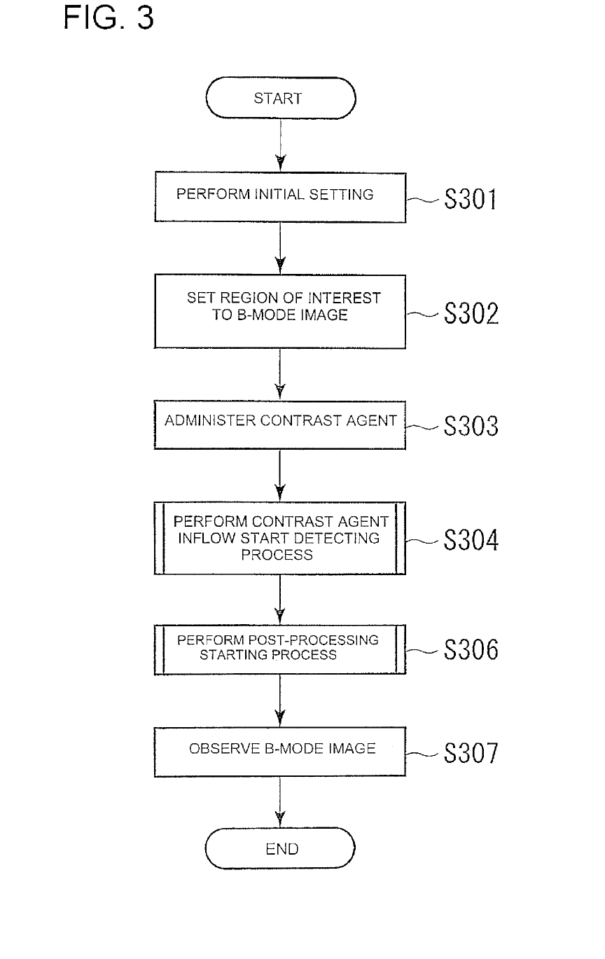

The operation of the controller 108 according to the embodiment 1 will next be explained using FIG. 3. FIG. 3 is a flowchart showing the operation of the controller 108. An operator first performs initial setting on the controller 108 (Step S301). Upon the initial setting, settings such as the selection of a B-mode, the startup of the contrast agent inflow start detecting means for automatically detecting the inflow of a contrast agent into the corresponding imaging region, a method for detection processing, the input of a threshold value, etc. are conducted from the input unit 107. Incidentally, the present embodiment 1 shows a case in which the average pixel value calculating means 63 is selected as the method for detection processing.

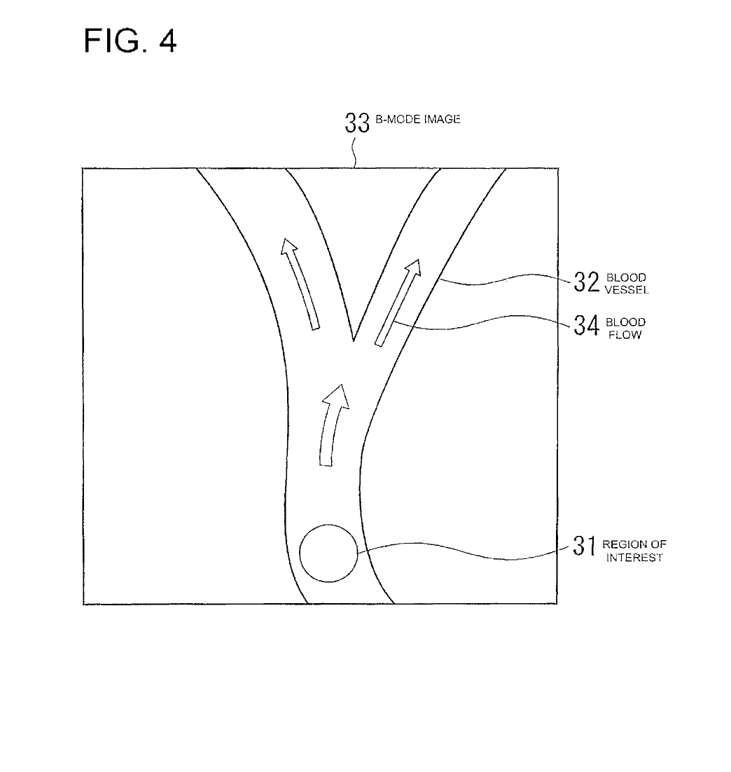

Thereafter, the operator brings the ultrasonic probe 10 into closely contact with the subject 2 and sets a region of interest (ROI) while causing the display unit 106 to draw or plot a B-mode image for a targeted imaging region (Step S302). Upon this setting of the region of interest, the region of interest is set to, for example, the corresponding blood vessel of the subject 2 drawn into the B-mode image of the display unit 106. FIG. 4 shows an example in which a region of interest 31 is set to a blood vessel 32 drawn into a B-mode image 33 displayed on the display unit 106. The region of interest 31 is set to a portion lying within the blood vessel 32 drawn into the B-mode image 33, at which the blood 34 flows in an imaging region.

Thereafter, the operator administers a contrast agent to the subject 2 (Step S303). The operator administers the contrast agent to a vein of the subject 2. After its administration, the contrast agent enters arteries from the vein via the heart and circulates in the body of the subject 2. With its circulation, the contrast agent moves into the arteries while remaining substantially in massive or agglomerated form and flows into various organs, e.g., the liver after a predetermined time interval. The contrast agent is gradually diffused while this circulation is being repeated and brought in the cells of a tissular portion such as the liver,

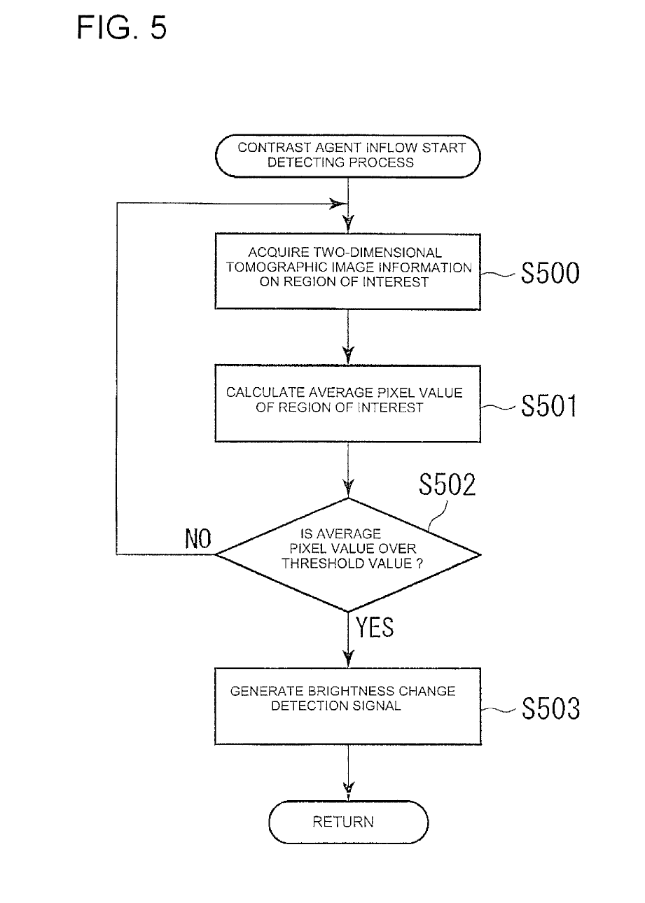

Thereafter, the controller 108 performs a contrast agent inflow start detecting process through the contrast agent inflow start detecting means 62 (Step S304). FIG. 5 is a flowchart showing the operation of the contrast agent inflow start detecting process executed by the contrast agent inflow start detecting means 62. In FIG. 5, a contrast agent inflow start is detected from a change in the average pixel value of the region of interest 31 set to the B-mode image 33. Now consider that the average pixel value calculating means 63 has been selected by the switch 69 in accordance with the initial setting conducted at Step S301.

The average pixel value calculating means 63 acquires the latest two-dimensional tomographic image information of the region of interest 31 set to the B-mode image 33 (Step S500) and calculates the average pixel value of pixel values contained in the two-dimensional tomographic image information (Step S501). The brightness change detecting means 66 compares the average pixel value with a threshold value set from the input unit 107 and determines whether it exceeds the threshold value (Step S502). When the average pixel value does not exceed the threshold value (NO at Step S502), the brightness change detecting means 66 proceeds to Step S500 and acquires two-dimensional tomographic image information of the region of interest 31 again. When the average pixel value has exceeded the threshold value (YES at Step S502), the brightness change detecting means 66 generates a brightness change detection signal assuming that the contrast agent is under inflow timing at which it has flowed in the corresponding imaging region (Step S503). The present contrast agent inflow start detecting process is terminated. Incidentally, the calculated average pixel value is stored in the unillustrated recording unit in sequence and forms information of a TIC to be described later.

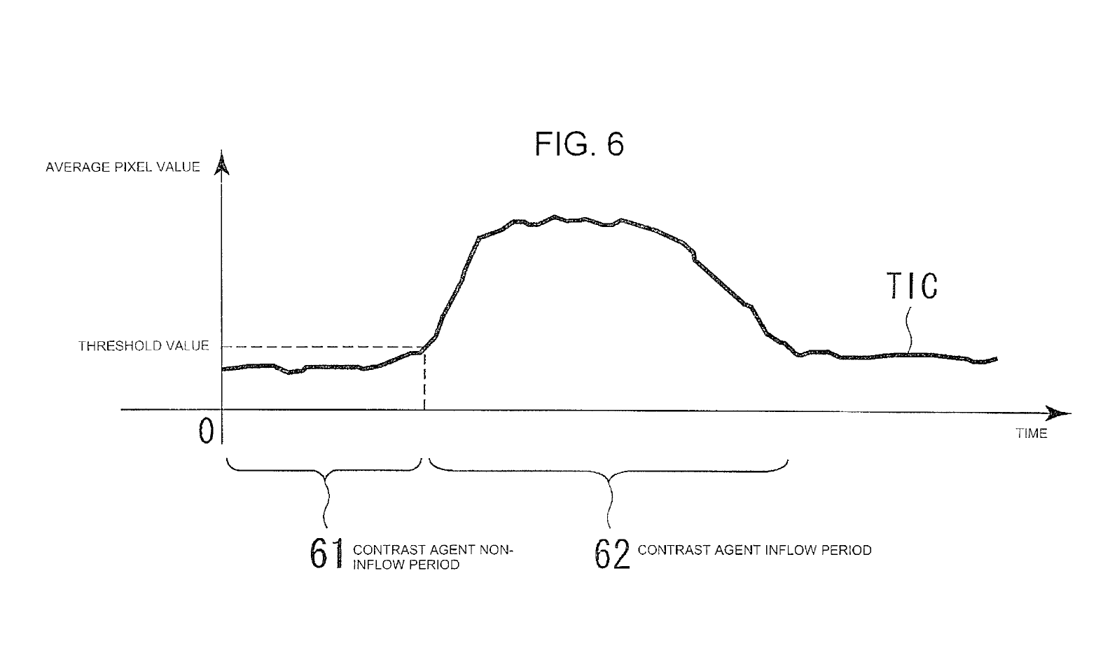

FIG. 6 illustrates by way of example, a TIC showing the manner in which the average pixel value of the region of interest 31 changes with time. The horizontal axis indicates the time, and the vertical axis indicates the average pixel value of the region of interest 31. Since the contrast agent is in a non-inflow state in the TIC initially, the TIC has a contrast agent non-inflow period 61 approximately constant at a low average pixel value.

When the contrast agent reaches the imaging region of the B-mode image 33, the contrast agent flows in the image of the blood vessel 32 as a high-brightness agglomerated region. The average pixel value of the region of interest 31 set to the inflow portion of the blood increases with the inflow of the contrast agent and forms a contrast agent inflow period 62. The contrast agent moves while being maintained substantially in an agglomerated state in the blood and passes through the region of interest 31. Along with it, the average pixel value is reduced again and assumes an average pixel value approximately similar to the contrast agent non-inflow period 61. Here, the threshold value for detecting the inflow of the contrast agent is experimentally decided so as to assume the minimum value that exceeds the range of variation in the average pixel value of the contrast agent non-inflow period 61.

Referring back to FIG. 3 subsequently, the post-processing starting means 68 starts a post-processing starting process subsequent to the flow of the contrast agent into the imaging region, based on the brightness change detection signal (Step S306).

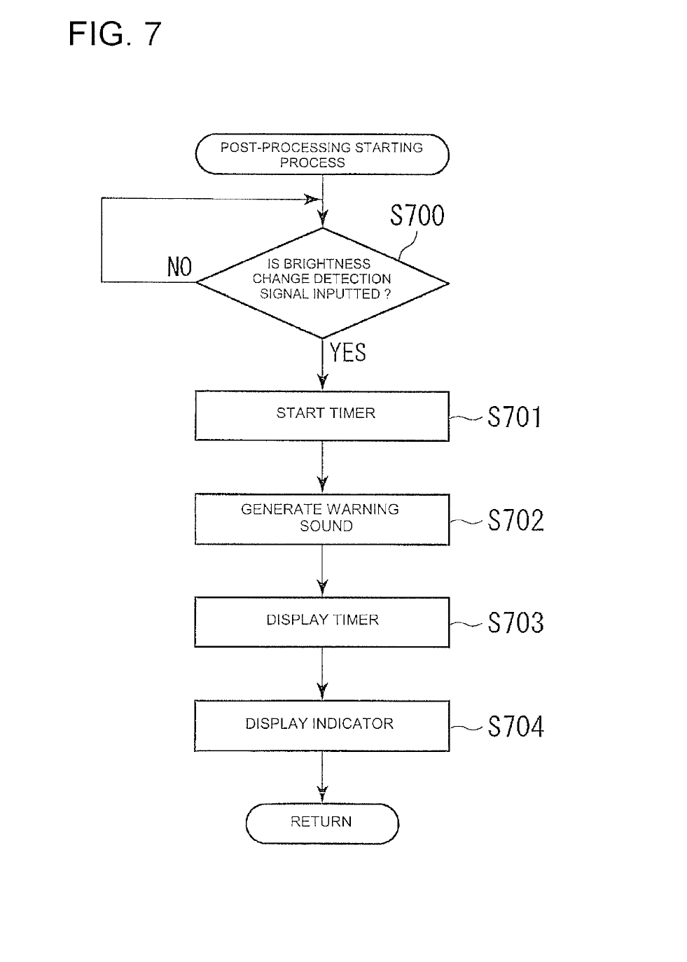

FIG. 7 is a flowchart showing the operation of the post-processing starting means 68. The post-processing starting means 68 first determines whether the brightness change detection signal is inputted (Step S700). When the brightness change detection signal is not inputted (NO at Step S700), the post-processing starting means 68 repeats this decision process until the brightness change detection signal is inputted.

When the brightness change detection signal is inputted (YES at Step S700), the post-processing starting means 68 starts the timer 700 assuming that the contrast agent is under inflow timing provided to start the inflow thereof in the imaging region (Step S701). Consequently, the timer 70 includes elapsed time information subsequent to the inflow of the contrast agent.

Thereafter, the post-processing starting means 68 produces a warning sound from the speaker 109 using the warning sound generating means 73 (Step S702). Owing to the warning sound, the operator recognizes the inflow of the contrast agent in the imaging region without observing the details of the B-mode image 33.

Afterwards, the post-processing starting means 68 starts the timer display control means 71 (Step S703). The timer display control means 71 transmits the elapsed time information of the timer 70 to the image display controller 105 with a predetermined time interval. The image display controller 105 displays the elapsed time information from the inflow of the contrast agent into the imaging region together with the B-mode image 33.

Thereafter, the post-processing starting means 68 starts the detected information display means 72 to cause the display unit 106 to display an indicator indicative of detected information (Step S704). The detected information display means 72 transmits information on the average pixel value calculated by the average pixel value calculating means 63 to the image display controller 105. The average pixel value information subsequent to the inflow of the contrast agent into the imaging region is displayed on the display unit 106 along with the B-mode image 33.

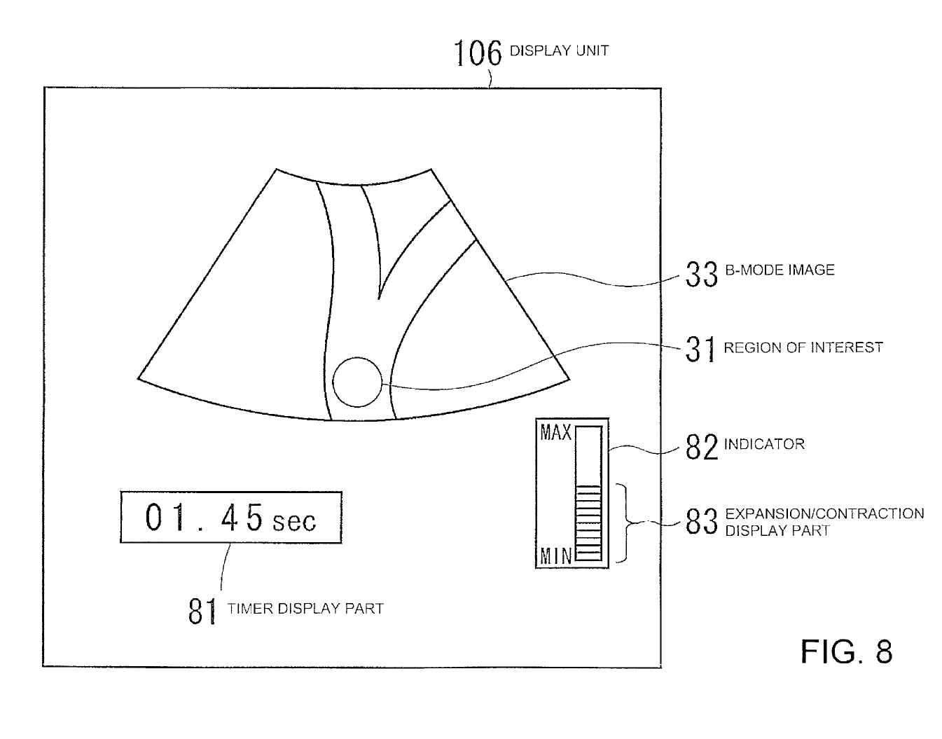

FIG. 8 is a diagram showing one example illustrative of elapsed time information and average pixel value information displayed along with a B-mode image 33. A tinier display part 81 and an indicator 82 arc contained in the display screen of the display unit 106 along with the B-mode image 33. The timer display part 81 numerically displays elapsed time information counted by the timer 70 in real time. The indicator 82 indicates the magnitude of an average pixel value of a region of interest 31 in the size of an expansion/contraction display part 83 that expands and contracts in the vertical direction. Incidentally, the indicator 82 can also indicate the magnitude of the average pixel value numerically.

Thereafter, the operator recognizes the inflow of the contrast agent into the imaging region according to a warning sound or a display start or the like of the timer display part 81 and observes the contrast agent drawn into the B-mode image 33 while making fine adjustments to the position or gain or the like of the ultrasonic probe 10 (Step S307), and terminates the actual process.

In the present embodiment 1 as mentioned above, the contrast agent inflow start detecting means 62 automatically detects the inflow of the contrast agent into the imaging region from the change in the brightness of the average pixel value contained in the region of interest 31 and subsequently automatically performs the generation of the warning sound, the startup and display of the timer 70, and the display of the indicator 82 indicative of the magnitude of the average pixel value. It is therefore possible to automatically start post-processing conducted after the inflow of the contrast agent into the imaging region, save operator's time and trouble and cause the operator to concentrate on the observation and optimization of the B-mode image 33 after the inflow of the contrast agent.

In the present embodiment 1, the average pixel value of the region of interest 31 is calculated using the average pixel value calculating means 63. It is determined that when the average pixel value exceeds the threshold value, the contrast agent has flowed in the imaging region. However, it is also possible to select the maximum pixel value calculating means 64 by the switch 69 in place of the selection of the average pixel value calculating means 63, calculate the maximum pixel value of the region of interest 31 and determine that the contrast agent has flowed in the imaging region when the maximum pixel value exceeds the threshold value. Incidentally, when the maximum pixel value calculating means 64 is selected, the displayed contents of the indicator 82 can be defined as information about the maximum pixel value.

In the present embodiment 1, the region of interest 31 is set to the B-mode image 33, and the inflow of the contrast agent is determined from the average pixel value or maximum pixel value of the region of interest 31. However, it is also possible to calculate the average pixel value or maximum pixel value using all two-dimensional tomographic image information about a B-mode image 33 containing all imaging regions and thereby determine the inflow of the contrast agent.

Although the present embodiment 1 has shown the example in which the timer display part 81 and the indicator 82 are displayed on the display unit 106, the time intensity curve (TIC) shown in FIG. 6 can also be displayed on the display unit 106 together with the B-mode image 33.

In the present embodiment 1, the post-processing starting means 68 has started up the warning sound generating means 73 and the timer 70 or the like. However, they can also be started up by recording a routine operation conducted after the contrast agent has flowed in the imaging region, e.g., a gain adjustment or the like as a micro program and synchronizing the micro program with the brightness change detection signal. Thus, the operator's routine operation conducted after the contrast agent has flowed in the imaging region can be further lightened.

Although the post-processing starting means 68 has started the warning sound generating means 73 in the present embodiment 1, it is also possible to light the lamp 101 based on the brightness change detection signal transmitted to the lamp lighting means 74 simultaneously with it or in place thereof and urge the operator to evoke his/her attention. Thus, the operator is able to recognize easier the inflow timing at which the contrast agent has flowed in the imaging region.

Incidentally, although, in the embodiment 1, the average pixel value or the maximum pixel value is calculated using the two-dimensional tomographic image information on the imaging region or the region of interest, and the inflow of the contrast agent is detected from the change in the brightness of each of these pixel values, it is also possible to calculate the histogram of each pixel value in the imaging region or the region of interest and detect the inflow of the contrast agent into the imaging region from the time change in the histogram. Therefore, an embodiment 2 will show a case in which a histogram of each pixel value is calculated from two-dimensional tomographic image information about an imaging region, and the inflow of a contrast agent into the imaging region is detected from a time change in the histogram.

Since the construction of an ultrasonic imaging apparatus 100 is exactly equal to one shown in FIGS. 1 and 2, its description is omitted here. Next, the operation of a controller 108 is exactly alike except for the initial setting of Step S301 in the flowchart shown in FIG. 3, the setting of the region of interest at Step S303 and the contrast agent inflow start detecting process of Step S304. Only different portions will be explained here.

Upon the initial setting of Step S301, an operator first performs a changeover setting of a switch 69 via an input unit 107 and selects a histogram calculating means 65.

Thereafter, the operator does not set the region of interest at Step S303. The calculation of a histogram to be described later is conducted using two-dimensional tomographic image information containing all imaging regions.

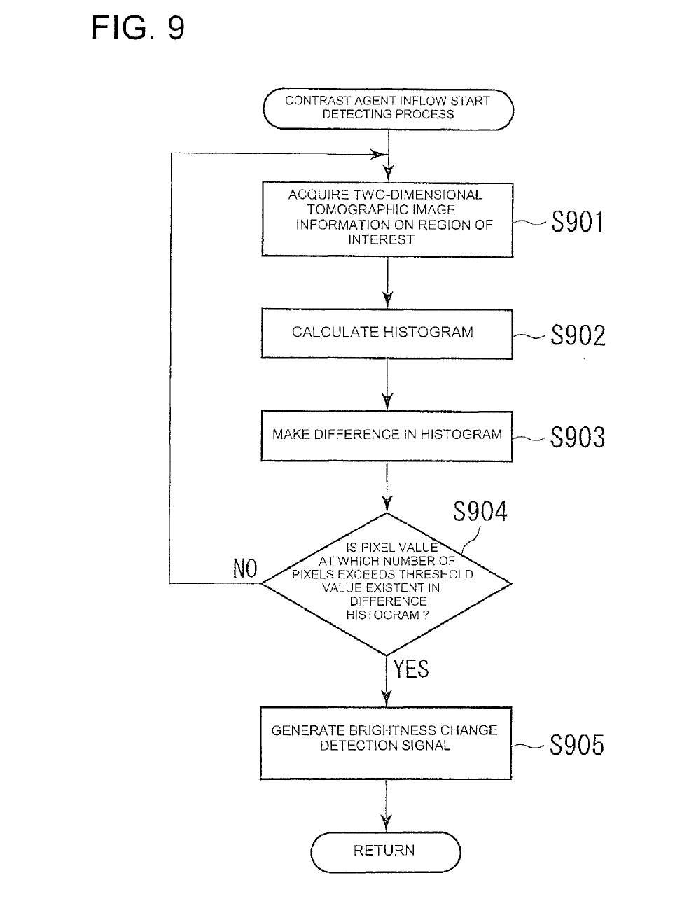

Afterwards, a contrast agent inflow start detecting means 62 of the controller 108 performs a contrast agent inflow start detecting process. FIG. 9 is a flowchart showing the operation of the contrast agent inflow start detecting process according to the present embodiment 2. The histogram calculating means 65 first acquires two-dimensional tomographic image information on the imaging region from an image memory 104 (Step S901).

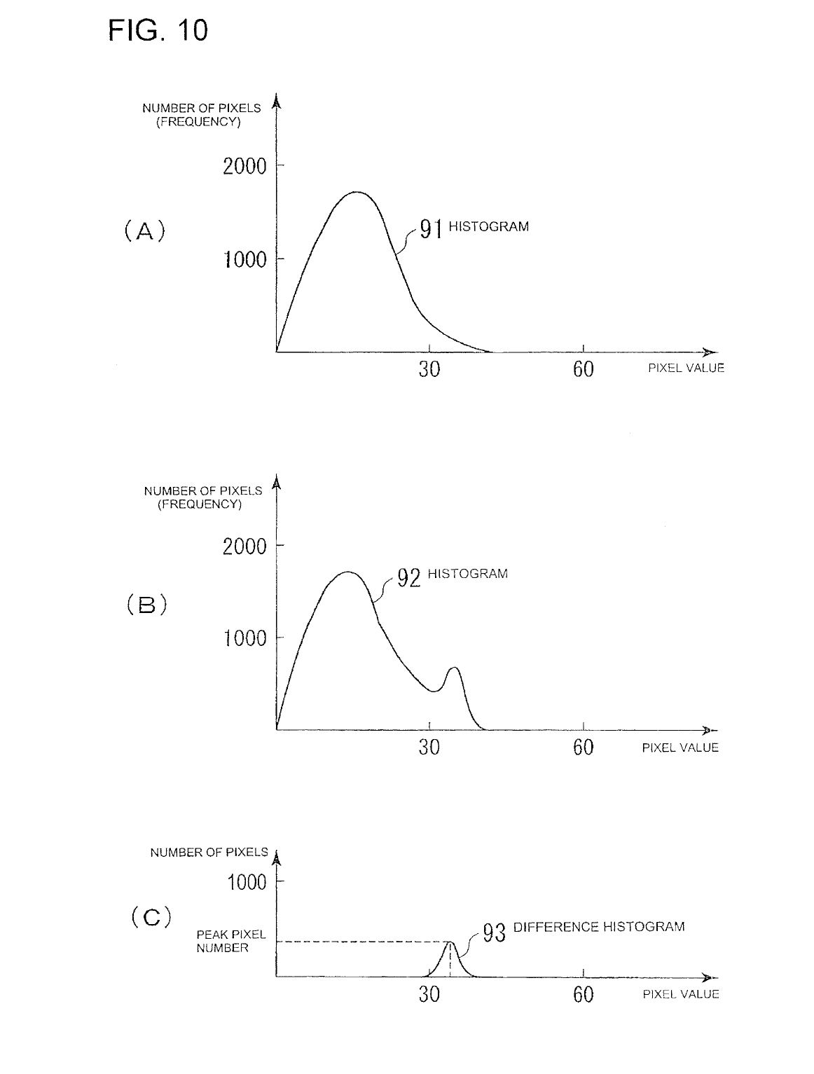

Subsequently, the histogram calculating means 65 calculates a histogram using the two-dimensional tomographic image information (Step S902). FIGS. 10(A), 10(B), and 10(C) are explanatory diagrams showing the histogram calculated using a B-mode image 33 by the histogram calculating means 65. FIG. 10(A) is a diagram of a histogram 91 where no contrast agent flows in an imaging region. The horizontal axis indicates a pixel value, and the vertical axis indicates the number of pixels (frequency). Incidentally, the values of the pixels illustrate all 64 levels of gray by way of example. A pixel value of the B-mode image 33 approximately exists between pixel values having gray levels equal to one half of zero to all gray levels. These pixel values arc values that a tissular portion of a subject 2 has.

FIG. 10(B) is a diagram of a histogram 92 where a contrast agent has flowed in an imaging region. In addition to a distribution represented by the pixels of the tissular portion shown in FIG. 10(A), a new peak occurs in a high pixel value portion with the inflow of the contrast agent. Incidentally, the histogram calculating means 65 has an unillustrated recording unit and stores therein information on the calculated histogram in time series. The information of the recording unit is used when a difference in histogram and the like to be described later are made.

Thereafter, the histogram calculating means 65 makes a difference between the calculated histogram and the histogram of the recording unit, which has been calculated prior to the former histogram and thereby calculates a difference histogram (Step S903). A brightness change detecting means 66 of the contrast agent inflow start detecting means 62 determines whether such a pixel value that the number of pixels exceeds a threshold value exists in the difference histogram (Step S904).

Here, when such a pixel value that the number of pixels exceeds the threshold value does not exist in the difference histogram (NO at Step S904), the brightness change detecting means 66 proceeds to Step S901 because no change occurs in the histogram and the contrast agent does not flow in the imaging region, and acquires two-dimensional tomographic image information on a succeeding frame.

When such a pixel value exists in the difference histogram (YES at Step S904), the brightness change detecting means 66 generates a brightness change detection signal since it is considered that a change occurs in the histogram and the contrast agent has flowed in the imaging region (Step S905).

FIG. 10(C) is an explanatory diagram showing a difference histogram 93 produced by making a difference between the histograms 91 and 92. In the difference histogram 93, only a pixel value portion at which a change in the number of pixels occurs due to the inflow of the contrast agent is extracted. The contrast agent flows in the blood vessels while an agglomerated state having a spread is being approximately maintained. The difference histogram 93 becomes one in which variations in the pixel value increase and spread as the spread of such an agglomerated portion becomes larger. When a peak pixel number at a peak contained in the difference histogram 93 exceeds a threshold value here, a contrast agent of predetermined dose is regarded to have been detected, and inflow timing at which the contrast agent has flowed in the corresponding imaging region, is reached. Thus, the brightness change detecting means 66 generates a brightness change detection signal.

In the present embodiment 2 as described above, the histogram calculating means 65 is capable of calculating the histograms from the two-dimensional tomographic image information on the imaging region and determining a change in the brightness of the imaging region from the difference between the histograms different in acquisition time.

Though the histograms for the imaging region have been determined by the histogram calculating means 65 in the present embodiment 2, it is also capable of setting a region of interest to the imaging region in like manner, determining histograms related to the region of interest and detecting a change in brightness.

Though the histograms for the imaging region have been determined by the histogram calculating means 65 in the present embodiment 2, it is also possible to cause the display unit 106 to display the histograms and visually determine the inflow of the contrast agent into the imaging region.

Many widely different embodiments of the invention may be configured without departing from the spirit and the scope of the present invention. It should be understood that the present invention is not limited to the specific embodiments described in the specification, except as defined in the appended claims.

1. An ultrasonic imaging apparatus comprising:
an image acquisition unit configured to acquire B-mode image information on an imaging region of a subject;
a display unit configured to display the B-mode image information;
a contrast agent inflow start detecting device configured to detect an inflow timing at which a contrast agent administered to the subject starts to flow in the imaging region, using the B-mode image information; and
a post-processing starting device configured to start post-processing in sync with the inflow timing after the contrast agent has started to flow in the imaging region.

2. The ultrasonic imaging apparatus according to claim 1, wherein said contrast agent inflow start detecting device comprises a brightness change detecting device configured to detect a brightness change that occurs in a B-mode image of the B-mode image information.

3. The ultrasonic imaging apparatus according to claim 2, wherein said brightness change detecting device is configured to set a timing for the brightness change related to the inflow timing.

4. The ultrasonic imaging apparatus according to claim 1, further comprising a region-of-interest setting device configured to set a region of interest to the B-mode image in the imaging region, which is displayed on said display unit.

5. The ultrasonic imaging apparatus according to claim 4, wherein said brightness change detecting device is configured to detect a brightness change in the B-mode image in the region of interest.

6. The ultrasonic imaging apparatus according to claim 5, wherein said brightness change detecting device comprises an average pixel value calculating device configured to calculate an average pixel value of a plurality of pixel values contained in the B-mode image.

7. The ultrasonic imaging apparatus according to claim 6, wherein said brightness change detecting device is configured to detect that the brightness change has occurred when the average pixel value exceeds a threshold value.

8. The ultrasonic imaging apparatus according to claim 6, wherein said average pixel value calculating device comprises a recording unit configured to store a time intensity curve indicative of a time change in the average pixel value.

9. The ultrasonic imaging apparatus according to claim 5, wherein said brightness change detecting device comprises a maximum pixel value calculating device configured to determine a maximum pixel value of a plurality of pixel values contained in the B-mode image information.

10. The ultrasonic imaging apparatus according to claim 9, wherein said brightness change detecting device is configured to determine that the brightness change has occurred when the maximum pixel value exceeds a threshold value.

11. The ultrasonic imaging apparatus according to claim 5, wherein said brightness change detecting device comprises a histogram calculating device configured to determine a histogram of pixel values contained in the B-mode image information.

12. The ultrasonic imaging apparatus according to claim 11, wherein said brightness change detecting device is configured to determine that the brightness change has occurred when a distribution of the histogram changes with time.

13. The ultrasonic imaging apparatus according to claim 6, wherein said brightness change detecting device further comprises a switch configured to select one of said average pixel value calculating device, said maximum pixel value calculating device, and said histogram calculating device.

14. The ultrasonic imaging apparatus according to claim 1, further comprising a speaker configured to produce a warning sound.

15. The ultrasonic imaging apparatus according to claim 14, wherein said post-processing starting device comprises a warning sound generating device configured to cause said speaker to produce the warning sound in sync with the inflow timing.

16. The ultrasonic imaging apparatus according to claim 1, further comprising a lamp configured to emit light for evoking attention.

17. The ultrasonic imaging apparatus according to claim 16, wherein said post-processing starting device comprises a lamp lighting device configured to cause said lamp to emit light in sync with the inflow timing.

18. The ultrasonic imaging apparatus according to claim 1, wherein said post-processing starting device comprises a timer configured to start counting in sync with the inflow timing.

19. The ultrasonic imaging apparatus according to claim 18, wherein said post-processing starting device further comprises a timer display control device configured to cause said display unit to display time information of said timer in sync with the inflow timing.

20. An ultrasonic imaging method comprising the steps of:
using B-mode image information acquired on an imaging region of a subject to detect an inflow timing at which a contrast agent administered to the subject starts to flow in the imaging region; and
starting up post-processing in sync with the inflow timing after the contrast agent has started to flow into the imaging region.
